(19) United States
(12) Patent Application Publication
(10) Pub. No.: US 2024/0374153 A1
(43) Pub. Date: Nov. 14, 2024

(54) ANCHORS AND WIRELESS SENSOR ATTACHMENT

(71) Applicants: W. L. Gore & Associates, Inc., Newark, DE (US); Integrated Sensing Systems, Inc., Ypsilanti, MI (US)

(72) Inventors: Kevin A. Gill, Flagstaff, AZ (US); Min-Hsuan Lin, Flagstaff, AZ (US); Rebecca L. Maryn, Flagstaff, AZ (US); David J. Minor, Phoenix, AZ (US); Edward E. Shaw, Flagstaff, AZ (US); Benjamin M. Trapp, Phoenix, AZ (US)

(21) Appl. No.: 18/686,929

(22) PCT Filed: Aug. 25, 2022
(86) PCT No.: PCT/US2022/041459

§ 371 Date: Feb. 27, 2024

Related U.S. Application Data
(60) Provisional application No. 63/237,867, filed on Aug. 27, 2021.

(51) Int. Cl. A61B 5/0215; A61B 5/00

(52) U.S. Cl. CPC A61B 5/0215; A61B 5/6847; A61B 5/6882

(57) ABSTRACT

Various aspects of the present disclosure are directed toward apparatuses. systems. and methods that include a catheter-delivered implantable hemodynamic monitor (IHM) implant system with an implantable sensor.

### CROSS-REFERENCE TO RELATED APPLICATION

This application is a national phase application of PCT Application No. PCT/US2022/041459, internationally filed on Aug. 25, 2022, which claims the benefit of Provisional Application No. 63/237,867, filed Aug. 27, 2021, which are incorporated herein by reference in their entireties for all purposes.

### FIELD

The present disclosure relates generally to apparatuses, systems, and methods that include coupling at least a portion of a sensor to an anchor. More particularly, the apparatuses, systems, and methods are directed toward a catheter-delivered implantable hemodynamic monitor (IHM) implant system with an implantable sensor placed on the atrial septum to monitor pressure in either left atrium or right atrium or both for the management of patients with congestive heart failure.

### BACKGROUND

Sensors may be implanted within a patient to acquire data. In certain instances, the sensor may be arranged or coupled to an anchor.

### SUMMARY

According to one example (“Example 1”), a sensor assembly for placement across the atrial septum of a patient, the sensor assembly comprising: a sensor having one or more recessed portions in an outer surface of the sensor; an anchor comprising: a first frame component; and a second frame component spaced from the first frame component, wherein the anchor is engaged with the one or more recessed portions in the outer surface of the sensor in a mating relationship such that the first frame component and the second frame component are rotationally fixed relative to the sensor.

According to another example (“Example 2”), further to the sensor assembly of Example 1, the anchor is engaged with the one or more recessed portions such that the first and second frame components are maintained at a selected spacing relative to one another.

According to another example (“Example 3”), further to the sensor assembly of Example 1, the anchor further comprises an engagement section coupled to at least one of the first frame component and the second frame component, the engagement section having an inner profile that is engaged with the one or more recessed portions in the outer surface of the sensor.

According to another example (“Example 4”), further to the sensor assembly of Example 3, the inner profile of the engagement section has a polygonal shape defining a plurality of engagement features for mating engagement with the one or more recessed portions in the outer surface of the sensor.

According to another example (“Example 5”), further to the sensor assembly of Example 3, the inner profile of the engagement section has a circular shape.

According to another example (“Example 6”), further to the sensor assembly of Example 3, the inner profile of the engagement section is defined by a membrane material.

According to another example (“Example 7”), further to the sensor assembly of any one of Examples 3-6, the engagement section is a first engagement section associated with the first frame component, and further wherein the anchor further comprises a second engagement section associated with the second frame component.

According to another example (“Example 8”), further to the sensor assembly of any one of Examples 3-7, the anchor further comprises one or more wires forming the first frame component, the second frame component and the engagement section, the inner circumference of the engagement section being defined by at least one turn of the one or more wires.

According to another example (“Example 9”), further to the sensor assembly of any one of Examples 1-8, the one or more recessed portions of the sensor include a first set of recessed portions and a second set of recessed portions, and the first frame component is coupled to the first set of recessed portions and the second frame component is coupled to the second set of recessed portions.

According to another example (“Example 10”), further to the sensor assembly of Example 1, the first frame component comprises a first plurality of engagement features defining a first engagement section of the anchor, and the second frame component comprises a second plurality of engagement features defining a second engagement section of the anchor, and further wherein the first plurality of engagement features engages the first set of recessed portions in a mating relationship and the second plurality of engagement features engages the second set of recessed portions in a mating relationship.

According to another example (“Example 11”), further to the sensor assembly of any one of Examples 1-5, each of the first frame component and the second frame component are convexly curved inwardly toward one another.

According to another example (“Example 12”), a sensor assembly for placement across the atrial septum of a patient, the sensor assembly comprising: a sensor having one or more recessed portions in an outer surface of the sensor; an anchor comprising: a first frame component; and a second frame component spaced from the first frame component, wherein the anchor is engaged with the one or more recessed portions in the outer surface of the sensor in a mating relationship such that the first frame component and the second frame component are maintained at a selected spacing relative to one another.

According to another example (“Example 13”), further to the sensor assembly of Example 12, the anchor further comprises a first engagement section associated with the first frame component that is configured to capture at least a portion of the sensor and engage the one or more recessed portions and a second engagement section associated with the second frame component that is configured to capture at least another portion of the sensor and engage the one or more recessed portions.

According to another example (“Example 14”), further to the sensor assembly of Example 13, the one or more recessed portions in the outer surface of the sensor includes a first set of recessed portions to which the first frame component is coupled and a second set of recessed portions to which the second frame component is coupled such that the first frame component and the second frame component are maintained at the selected spacing relative to one another.

According to another example (“Example 15”), further to the sensor assembly of any one of Examples 12-14, the first and second engagement sections each comprise at least one wire.

According to another example (“Example 16”), further to the sensor assembly of Example 15, the at least one wire includes a continuous wind pattern through the first frame component and the second frame component.

According to another example (“Example 17”), further to the sensor assembly of Example 13, the first and second engagement sections are engaged with the one or more recessed portions in the outer surface of the sensor in a mating relationship such that a rotational orientation of the first and second frame components is maintained.

According to another example (“Example 18”), a method of managing treatment of a patient with congestive heart failure including arranging a first frame component of an anchor on a first side of the atrial septum, the anchor being in mating engagement with a plurality of recessed portions in an outer surface of a sensor; and arranging a second frame component of the anchor on a second side of the atrial septum such that the sensor is arranged to monitor pressure in the left atrium, the right atrium, or both the left atrium and the right atrium.

According to another example (“Example 19”), further to the method of Example 18, the anchor comprises an engagement section coupled to at least one of the first frame component and the second frame component, and further wherein the engagement section is in mating engagement with the plurality of recessed portions in the outer surface of the sensor such that a rotational orientation of the first and second frame components relative to the sensor is maintained.

According to another example (“Example 20”) further to the method of Examples 18 or 19, the engagement section is in mating engagement with the plurality of recessed portions in the outer surface of the sensor such that a spacing between the first and second frame components is maintained.

According to another example (“Example 21”), further to the method of any one of Examples 18-20, the plurality of recessed portions of the sensor include a first set of recessed portions and a second set of recessed portions, and the first frame component is coupled to the first set of recessed portions and the second frame component is coupled to the second set of recessed portions.

According to another example (“Example 22”), a sensor assembly for placement across the atrial septum of a patient including a sensor having one or more recessed portions in an outer surface of the sensor; a first frame component including a plurality of first contact features and a first engagement section configured to capture at least a portion of the sensor and engage the one or more recessed portions to limit rotational freedom of the sensor relative to the first frame component; and a second frame component including a plurality of second contact features and a second engagement section configured to capture at least a portion of the sensor and engage the one or more recessed portions to limit rotational freedom of the sensor relative to the second frame component.

According to another example (“Example 23”), further to the sensor assembly of Example 22, the first engagement section is defined by a plurality of first inner apices that approximate a shape that includes a first circumference that is about equal to a circumference of the sensor and the second engagement section is defined by a plurality of second inner apices that approximate a shape that includes a second circumference that is about equal to a circumference of the sensor.

According to another example (“Example 24”), further to the sensor assembly of Examples 22 or 23, the first and second engagement sections are configured to maintain a spacing between the first and second frame components.

According to another example (“Example 25”), further to the sensor assembly of any one of Examples 22-24, each of the first frame component and the second frame component are convexly curved toward one another.

According to another example (“Example 26”), further to the sensor assembly of Example 23, the one or more recessed portions in the exterior surface of the sensor are configured to engage with the plurality of first inner apices and the plurality of second inner apices.

According to another example (“Example 27”), a sensor assembly for placement across the atrial septum of a patient includes a sensor having one or more recessed portions in an outer surface; a first frame component including a plurality of first contact features; a second frame component including a plurality of second contact features; and an intermediate portion including a graft material arranged between and connecting the first frame component and the second frame component, the intermediate portion being configured to engage the one or more recessed portions in a mating relationship to couple the first and second frame components to the sensor.

According to another example (“Example 28”), further to the sensor assembly of Example 27, the first frame component includes a plurality of first inner apices that approximate a shape that includes a first circumference that is greater than a circumference of the sensor and the second frame component includes a plurality of second inner apices that approximate a shape that includes a second circumference that is greater than a circumference of the sensor.

According to another example (“Example 29”), a sensor assembly for placement across the atrial septum of a patient including a sensor having one or more recessed portions in an outer surface; a first frame component including a plurality of first contact features and a first engagement section forming a first inner boundary configured to engage the recessed portions in mating engagement to limit rotational freedom of the sensor relative to the first frame component; and a second frame component arranged opposite the first frame component on the sensor, the second frame component including a plurality of second contact features and a second engagement section forming a second inner boundary configured to engage the recessed portions in mating engagement to limit rotational freedom of the sensor relative to the second frame component.

According to another example (“Example 30”), further to the sensor assembly of Example 29, the first frame component and the second frame component are convexly curved inwardly toward a center of the anchor between the first frame component and the second frame component to maintain a space between one another when the sensor assembly is operatively deployed in a patient.

According to another example (“Example 31”), further to the sensor assembly of Example 29, further including an intermediate portion including a graft material arranged between and connecting the first frame component and the second frame component.

According to another example (“Example 32”), further to the sensor assembly of Example 29, the first engagement section has an approximately circular shape and the second engagement section has an approximately circular shape.

According to one example (“Example 33”) a sensor assembly for placement across the atrial septum of a patient including a sensor having one or more recessed portions in an outer surface of the sensor; a first frame component including a first engagement section forming a boundary configured to capture at least a portion of the sensor and engage the one or more recessed portions; and a second frame component including a second engagement section forming a boundary configured to capture at least a portion of the sensor and engagement engage the one or more recessed portions, the first and second engagement sections being configured to maintain a space between the first and second frame components.

According to another example (“Example 34”), further to the sensor assembly of Example 33, the first frame component and the second frame component are curved inwardly toward a center of the anchor between the first and second frame components such that the pluralities of first and second contact features convexly curve toward one another.

According to another example (“Example 35”), further to the sensor assembly of Examples 33 or 34, the first engagement section has an approximately polygonal shape and the second engagement section has an approximately polygonal shape.

According to one example (“Example 36”), a sensor assembly for placement across the atrial septum of a patient includes a sensor having one or more recessed portions in an outer surface; an anchor comprising: an engagement section configured to engage the one or more recessed portions in the sensor with an interference fit to hold the sensor within the anchor; and a first frame component and a second frame component, each of the first frame component and the second frame component being convexly curved inwardly toward the engagement section to maintain a space between the first and second frame components.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is an example anchor for coupling to a sensor in accordance with an embodiment.

FIG. 2 is an example frame component that may form a part of an anchor for coupling to a sensor in accordance with an embodiment.

FIG. 3 is an example frame component that may form a part of an anchor for coupling to a sensor and a winding configuration in accordance with an embodiment.

FIG. 4 is an example anchor for coupling to a sensor in accordance with an embodiment.

FIG. 5 is an example device that may be arranged with an anchor in accordance with an embodiment.

FIG. 6A is another example anchor for coupling to a sensor in accordance with an embodiment.

FIG. 6B is a side view of the anchor, shown in FIG. 6A, for coupling to a sensor in accordance with an embodiment.

FIG. 7 is another example anchor for coupling to a sensor in accordance with an embodiment.

FIG. 8 is another example anchor for coupling to a sensor in accordance with an embodiment.

FIG. 9 is another example anchor for coupling to a sensor in accordance with an embodiment.

FIG. 9 is another example anchor for coupling to a sensor in accordance with an embodiment.

FIG. 10A is an example anchor and a sensor coupled to the anchor in accordance with an embodiment.

FIG. 10B is an example anchor and a sensor coupled to the anchor in accordance with an embodiment.

FIG. 10C is an example anchor and an implantable medical device coupled to the implantable medical device in accordance with an embodiment.

FIG. 11 is an example sensor having engagement sections spaced about a circumference of the sensor in accordance with an embodiment.

FIG. 12 is another example sensor having engagement sections spaced about a circumference of the sensor in accordance with an embodiment.

FIG. 13 is an example anchor and a sensor coupled to the anchor in accordance with an embodiment.

FIGS. 14A-C show example perimeters for an intermediate section configured to engage a sensor in accordance with an embodiment.

FIGS. 15A-B are perspective and side views of an example anchor configured to engage a sensor in accordance with an embodiment.

FIG. 16 is an example anchor and a sensor coupled to the anchor in accordance with an embodiment.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

### Description of Various Embodiments

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

Various aspects of the present disclosure are directed toward arranging an implantable sensor within a patient. In certain instances, the implantable sensor may be arranged with and/or coupled to an anchor. The implantable sensor, which may be a wireless pressure sensor, may monitor heart failure and titrate medical therapy to prevent heart failure hospitalizations. In certain instances, the sensor may be arranged within a septum of the heart and can access one or both left and right atrial pressures. Thus, the sensor is delivered and anchored to the septum. The anchors, as discussed herein, may facilitate anchoring the sensor and minimize risk of embolization, minimize risk of creating loose thrombus, and/or minimize risk of enabling loose tissue growth—all of which can cause stroke. Further, the anchors, as discussed herein, may arrange the sensor or sensor housing to protrude beyond the septal wall to avoid the tissue overgrowth over the sensor face (which may disturb the pressure reading). The apparatuses, systems, and methods are directed toward a catheter-delivered implantable hemodynamic monitor (IHM) implant system with an implantable sensor placed on the atrial septum to monitor pressure in either left atrium or right atrium or both for the management of patients with congestive heart failure.

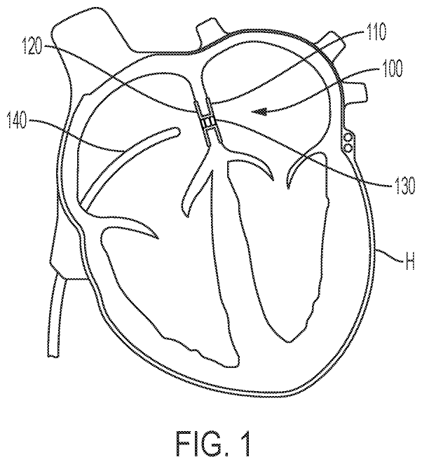

FIG. 1 is an example anchor for measuring blood pressure or pressures in accordance with an embodiment. The anchor 100 is shown implanted within a heart H of a patient. The device 100 is shown arranged between the patient's left atrium and right atrium. In certain instances, the device 100 may be used to measure blood pressure or pressures within the heart H, for example, between the left and right atria LA, RA. As shown, the device 100 generally includes a first frame component 110 arranged on a first side of a septum (e.g., within the right atrium RA), a second frame component 120 arranged on a second side of the septum (e.g., within the left atrium LA), and an engagement section 130 extending through the septum. A needle may be used to create an opening in the septum.

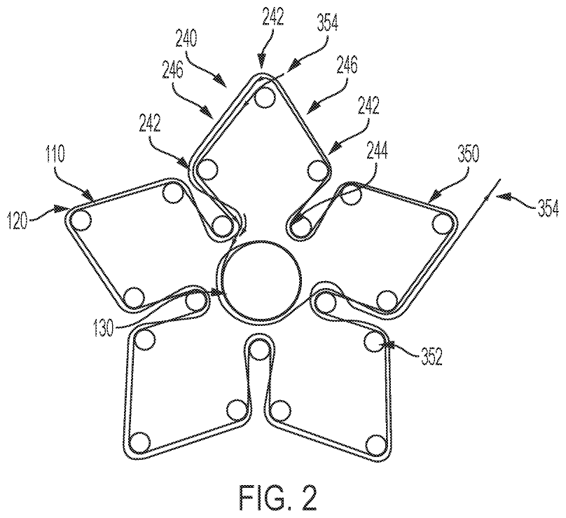

A sheath 140 and constraining and/or release lines (not shown) may be used to facilitate deployment of the device 100. For example, a first side of the device 100 that includes the first frame component 110 may be released after the sheath 140 is advanced through the septum and to the LA, and the second frame component 120 that includes the second frame component 120 may be released on the RA side of the septum. An engagement section 130 (e.g., shown in FIG. 2) is arranged within the opening. The frame components 110, 120 and/or the engagement section 130 may be compressed within the sheath 140 during delivery of the device 100 to the desired treatment area within the patient and subsequently expanded during deployment of the device 100. In other instances, the anchor 100 may be implanted at a different location in the heart or a different location within the patient (e.g., gastrointestinal system, vasculature, brain).

In certain instances, the engagement section 130 is configured to engage at least a portion of a sensor. The engagement section 130, in certain instances, may extend beyond each of the first frame component 110 and the second frame component 120 and between the first frame component 110 and the second frame component 120 to facilitate positioning of the sensor.

FIG. 2 shows an example winding pattern of first frame component 110 and second frame component 120. In particular, FIG. 2 shows two passes of a wire about a winding pattern to define the first frame component 110 and the second frame component 120, as well as engagement section 130 between the first and second frame components. The first frame component 110 is shown as the inner winding and the second frame component 120 is slightly offset and shown as an outer winding. This is done for illustrative purposes, as this is a two-dimensional representation of a three-dimensional object, and the windings may have essentially the same size according to various embodiments. And, although both the first and second frame components 110, 120 are shown, only one frame component may take the general shape shown in FIG. 2, and the other may be absent, or take a different shape according to various examples. As shown, a single wire 350 may be wound about a plurality of winding pegs 352 to form the first frame component 110 (and second frame component 120) as desired.

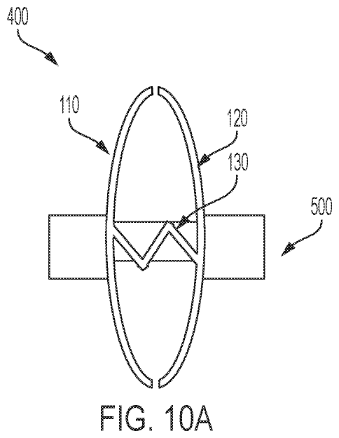

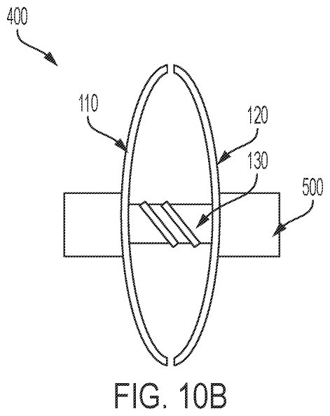

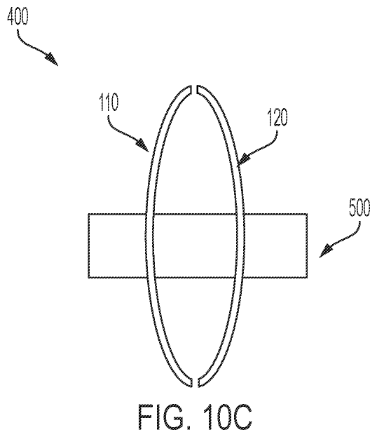

First frame component 110 (and second frame component 120) may form a part of an anchor for coupling to at least a portion of a sensor in accordance with an embodiment. The shape shown for the first frame component 110 and/or the second frame component 120 may be the shape and configuration of the first frame component 110 and/or the second frame component 120 shown in FIGS. 1, 10A-C. As shown, the first frame component 110 may include one or more wires, as can the second frame component 120. As described in further detail below, the one or more wires 350 may form the first frame component 110, the second frame component 120, and an engagement section 130 (e.g., as shown in FIGS. 1, 10A-C) of an anchor for coupling to a sensor. The first frame component 110 may be arranged on a first side of the engagement section 130 and the second frame component 120 may be arranged on a second side of the engagement section 130.

Each of the first frame component 110 and the second frame component 120 may include a plurality of contact features 240, one location for which is highlighted in FIG. 2 for ease of illustration. In instances where an implantable device includes the first frame component 110 and the second frame component 120, the contact features 240 of the first frame component 110 may be a first plurality of contact features 240 and the contact features 240 of the second frame component 120 may be a second plurality of contact features 240. The contact features 240, in certain instances, may include a plurality of outer apices 242 and at least one inner apex 244 arranged between each of the plurality of contact features 240. In instances where an implantable device includes the first frame component 110 and the second frame component 120, the plurality of outer apices 242 and the at least one inner apex 244 of the first frame component 110 may be a first plurality of outer apices 242 and at least one first inner apex 244 and the plurality of outer apices 242 at least one inner apex 244 of the second frame component 120 may be a plurality of second outer apices 242 and at least one second inner apex 244.

In certain instances, the one or more wires 350 that form the frame component 100 include a substantially linear section 246 between each of the plurality of outer apices 242. The substantially linear section 246 between each of the plurality of outer apices 242 may form approximately a diamond shape of the contact features 242 as is shown in FIG. 2. In addition, the plurality of contact features 242 may be an odd number of contact features 242 (e.g., three, five, seven) in certain instances or an even number of contact features 242 (e.g., two, four, six, eight) in other instances. In certain instances, the at least one inner apex 244 of each of the plurality of contact features 242 is adjacent to and spaced about a circumference of the engagement section 130. In certain instances, the outer apices 242 and at least one inner apex 244 have curvatures. In certain instances, the portions of the one or more wires 350 that form the first frame component 110 are uncoupled from portions of the one or more wires 350 that form the second frame component 120.

In certain instances, the plurality of outer apices 242 may be used to engage deployment tethers or portions of a deployment system. The plurality of outer apices 242 may also engage retrieval loops to allow for recapture or retrieval of the device after it is deployed. The deployment system may use one or more constraining lines and/or release lines as discussed in further detail in WO2020018697A1 (Cole et al.) which is incorporated herein.

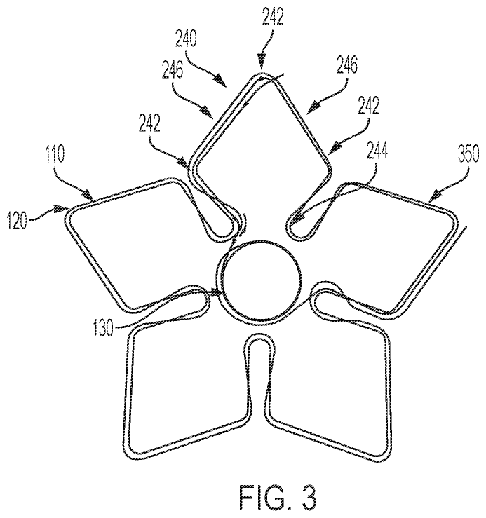

FIG. 3 shows the first frame component 110 and the second frame component 120 that may form a part of an anchor for coupling to a sensor and a winding configuration in accordance with an embodiment. In FIG. 3, the winding pegs 352 are not shown, and the ends of wire 350 have been trimmed to the final frame shape. Though one wire is shown in FIGS. 2 and 3, the first frame component 110 and/or second frame component 120 may be formed of one or more wires 350

As shown in FIG. 2, in certain instances, the wire(s) 350 may including a starting point 354 and an ending point 354 in forming a first frame component 110. In certain instances, the starting point 354 of the wire(s) 350 may be within the first frame component 110 and the ending point 354 may be within a second frame component 120. In addition, the wire(s) 350 may form the engagement section 130. In certain instances, the engagement section 130 includes a circumference that includes at least one turn of the wire(s) 350. The wire(s) 350 may include at least one wire 350 forming the first plurality of contact features 242, the engagement section 130, and the second plurality of contact features 242. The wire(s) 350 includes a continuous wind pattern in forming the first frame component 110, the second frame component 120, and the engagement section 130.

As shown, the first frame component 110 includes five contact features 242 each of which include a diamond shape. In certain instances, the five contact features 242 (or an odd number of contact features) may resist prolapse when implanted in the atrial septum (or target area of the body). The shape and arrangement of the contact features 242 may resist prolapse and stabilize a device arranged within the engagement section 130. The odd number of contact features 242 and shape of the contact features may facilitate crush or constraining of the contact features 242 while also minimizing stress, minimizing prolapse, and allowing for retrievability. In certain instances, the frame components 110, 120 may be substantially planar or may be concave or convex. In certain instances, the portions of the one or more wires 350 that form the first frame component 110 are uncoupled from portions of the one or more wires 350 that form the second frame component 120.

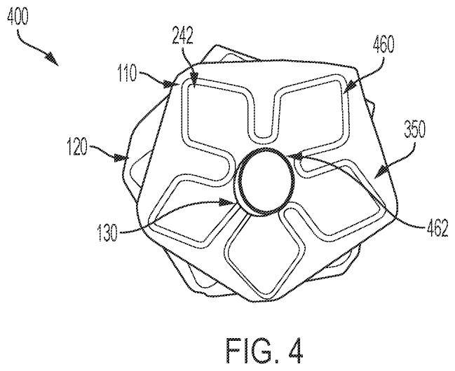

FIG. 4 is an example anchor 400 for coupling to a sensor in accordance with an embodiment. The anchor 400 may be part of a system that includes a sensor (or other device to be arranged with or coupled to the anchor 400). As discussed in detail above, the anchor 400 includes an engagement section 130 configured to capture the sensor within the engagement section, a first frame component 110 arranged on a first side of the engagement section 130, which may include an odd number of contact features 242 each having a portion with an approximate diamond shape, and a second frame component 120 arranged on a second side of the engagement section 130, which may include an odd number of contact features 242 each having a portion with an approximate diamond shape.

In certain instances, the first frame component 110, the second frame component 120, and the engagement section 130 are formed by at least one wire. As shown in FIG. 3, the wire(s) 350 includes a continuous wind pattern in forming the first frame component 110, the second frame component 120, and the engagement section 130. In certain instances, the first frame component 110 and the second frame component 120 are formed from separate wires 350 with separate membranes. The first frame component 110 and the second frame component 120 may be separate from one another. In other instances, the engagement section 130 may be formed of a membrane that separates the first frame component 110 and the second frame component 120. In addition, one or both of the first frame component 110 and the second frame component 120 may be concave.

In certain instances and as is shown in FIG. 4, the contact features 242 of the first frame component 110 and the contact features 242 of the second frame component 120 are aligned (e.g., relative to a longitudinal axis of the engagement section 130). The contact features 242 of each of the frame components 110, 120 being allowed may allow for crossing or re-crossing of the anchor 400 after implantation. In certain instances, the anchor 400 includes a membrane 460 coupled to the first frame component 110 and the second frame component 120. The membrane 460 may be arranged across the contact features 242 of the first frame component 110 and the contact features 242 of the second frame component 120. As shown in FIG. 4, the membrane 460 spans areas 462 of the first frame component 110 and the second frame component 120 that do not include the at least one wire that forms the first frame component 110 and the second frame component 120. As noted above, the contact features 242 of the first frame component 110 and the contact features 242 of the second frame component 120 are aligned. The areas 462 where there is no wire(s) may be punctured for recrossing (e.g., for a future procedure such as ablation, left atrial appendage device implantation).

In implanting the anchor 400, which may be used in a method of managing treatment of a patient with congestive heart failure, the anchor 400 may be placed on a tissue (e.g., atrial septum) of the patient. The first frame component 110 may be arranged on a first side of the septum and the second frame component 120 may be arranged on a second side of the septum. After deployment of the anchor 400, a sensor may be arranged with the engagement section 130 to monitor pressure in either left atrium or right atrium or both. In other instances, the sensor may be built with the anchor 400 and delivered in place. In certain instances, the portions of the one or more wires 350 that form the first frame component 110 are uncoupled from portions of the one or more wires 350 that form the second frame component 120.

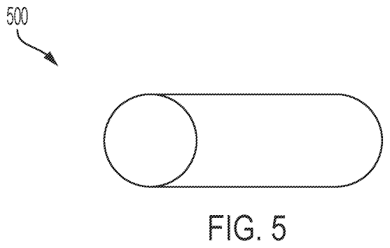

FIG. 5 includes a generalized view of a sensor 500 that may be coupled to the implantable device 400 and anchored within the body of a patient.

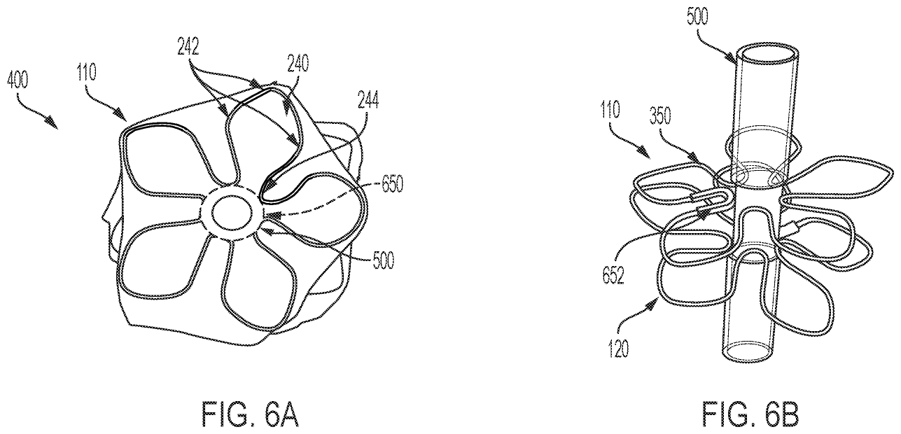

FIG. 6A is another example anchor 400 for coupling to a sensor in accordance with an embodiment. The anchor 400 may anchor a sensor within a patient. The first frame component 110 shown in FIG. 6A may be the first frame component 110 and/or the second frame component 120 (e.g., as shown in FIGS. 1, 10A-C). The first frame component 110 may include one or more wires. The one or more wires 350 may form the first frame component 110, the second frame component 120, and an intermediate portion (e.g., engagement section shown in FIG. 1). In other instances, the intermediate portion may include a graft material arranged between and connecting the first frame component 110 and the second frame component 120. The first frame component 110 may be arranged on a first side of the intermediate portion and the second frame component 120 may be arranged on a second side of the intermediate portion.

The first frame component 110 may include a plurality of contact features 240, one of which is highlighted in FIG. 6A for ease of illustration. In instances where an implantable device includes the first frame component 110 and the second frame component 120, the contact features 240 of the first frame component 110 may be a first plurality of contact features 240 and the contact features 240 of the second frame component 120 may be a second plurality of contact features 240. The contact features 240, in certain instances, may include a plurality of outer apices 242 and a plurality of inner apices 244 (or contact features 244) arranged between each of the plurality of contact features 240. In instances where an implantable device includes the first frame component 110 and the second frame component 120, the plurality of outer apices 242 and the plurality of inner apices 244 of the first frame component 110 may be a first plurality of outer apices 242 and the plurality of inner apices 244 and the plurality of outer apices 242 and the plurality of inner apices 244 of the second frame component 120 may be a plurality of second outer apices 242 and a plurality of second inner apices 244. In certain instances, a single frame component 110 may be used to anchor a sensor 500 with the adjacent ones of the contract features 240 being arranged on opposite sides of tissue.

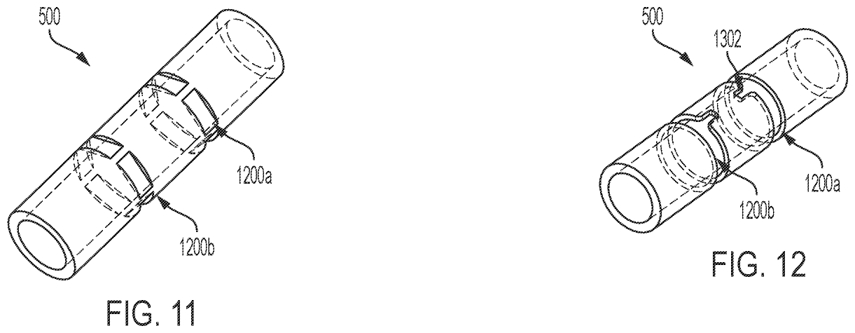

The inner apices 242, 244 (or contact features 242, 244) may each serve as engagement features for engagement (e.g., mating engagement) with an outer surface of a sensor. In certain instances and as is shown, the plurality of first inner apices 242 and/or the plurality of second inner apices 244 may be configured to capture at least a portion of a sensor 500 (e.g., by engaging recessed portions in the sensor 500 as described in detail below with reference to FIGS. 11-12). The apices 242, 244 may be engagement features. The plurality of first inner apices 244 may approximate a shape that includes a first circumference 650 that is about equal to a circumference of the sensor 500 and/or the plurality of second inner apices 244 may approximate a shape that includes a second circumference 650 that is about equal to a circumference of the sensor. The plurality of first inner apices 244 and/or the plurality of second inner apices 244 may be configured to capture at least a portion of the sensor 500 to hold and anchor the sensor 500 in place within the anchor 400 (e.g., by engaging recessed portions in the sensor 500 as described in detail below with reference to FIGS. 11-12). The plurality of first inner apices 244 and/or the plurality of second inner apices 244 may be configured to limit rotational freedom of the sensor 500 when anchored within the anchor 400.

In certain instances, the portions of the one or more wires 350 that form the first frame component 110 are uncoupled from portions of the one or more wires 350 that form the second frame component 120.

FIG. 6B is a side view of the anchor 400, shown in FIG. 6A, for coupling to a sensor in accordance with an embodiment. As shown, the frame components 110, 120 are isolated from one another and do not include a membrane to show the wires 350 that form the frame components 110, 120. In certain instances, the frame components 110, 120 may each be formed by a single wire 350. In other instances, a single wire may form both of the frame components 110, 120. Further, multiple wires 350 may form both the frame components 110, 120 or multiple separate wires 350 may be used to individually form the frame components 110, 120. In certain instances, a graft section 652 may be used to hold together ends of the wires 350 used to form the frame components 110, 120. The ends of the wires 350 or wire 350 not touching may lessen or eliminate signal interference for the sensor 500.

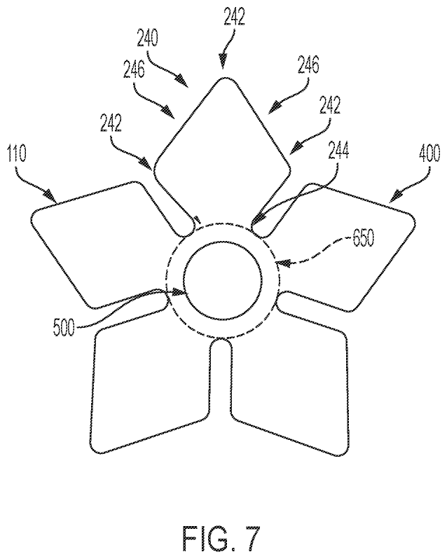

FIG. 7 is another example anchor 400 for coupling to a sensor in accordance with an embodiment. The anchor 400 may anchor a sensor within a patient. The first frame component 110 shown in FIG. 7 may be the first frame component 110 and/or the second frame component 120 (e.g., as shown in FIGS. 1, 10A-C). The first frame component 110 may include one or more wires. The one or more wires 350 may form the first frame component 110, the second frame component 120, and an intermediate portion (e.g., engagement section shown in FIG. 1). In other instances, the intermediate portion may include a graft material arranged between and connecting the first frame component 110 and the second frame component 120. The first frame component 110 may be arranged on a first side of the intermediate portion and the second frame component 120 may be arranged on a second side of the intermediate portion.

The first frame component 110 may include a plurality of contact features 240, one of which is highlighted in FIG. 7 for ease of illustration. In instances where an implantable device includes the first frame component 110 and the second frame component 120, the contact features 240 of the first frame component 110 may be a first plurality of contact features 240 and the contact features 240 of the second frame component 120 may be a second plurality of contact features 240. The contact features 240, in certain instances, may include a plurality of outer apices 242 and a plurality of inner apices 244 arranged between each of the plurality of contact features 240. In instances where an implantable device includes the first frame component 110 and the second frame component 120, the plurality of outer apices 242 and the plurality of inner apices 244 of the first frame component 110 may be a plurality of first outer apices 242 and a plurality of first inner apices 244 and the plurality of outer apices 242 and the plurality of inner apices 244 of the second frame component 120 may be a plurality of second outer apices 242 and a plurality of second inner apices 244. In certain instances, a single frame component 110 may be used to anchor a sensor 500 with the adjacent ones of the contract features 240 being arranged on opposite sides of tissue.

The inner apices 242, 244 (or contact features 242, 244) may each serve as engagement features for engagement (e.g., mating engagement) with an outer surface of a sensor. The plurality of first inner apices 244 may approximate a shape that includes a first circumference 650 that is greater than a circumference of the sensor 500 and/or the plurality of second inner apices 244 may approximate a shape that includes a second circumference 650 that is greater than a circumference of the sensor. The first circumference 650 may be configured to limit rotational freedom of the sensor 500 when anchored within the anchor 400.

In certain instances, the portions of the one or more wires 350 that form the first frame component 110 are uncoupled from portions of the one or more wires 350 that form the second frame component 120. In addition, the first frame component 110 and the second frame component 120 may be separated by an intermediate portion (e.g., as shown in FIGS. 1, 10A-C). The intermediate portion may be formed by portions of the wires 350 and/or by a graft material arranged between and connecting the first frame component 110 and the second frame component 120. The intermediate portion may be configured to engage and capture a sensor (e.g., as shown in FIGS. 10 and 11).

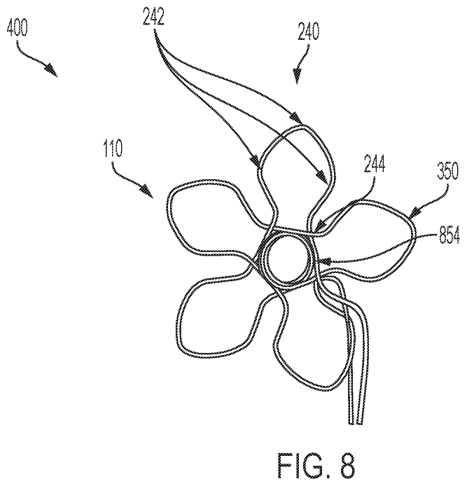

FIG. 8 is another example anchor for coupling to a sensor in accordance with an embodiment. The anchor 400 may anchor a sensor within a patient. The first frame component 110 shown in FIG. 8 may be the first frame component 110 and/or the second frame component 120 (e.g., as shown in FIGS. 1, 10A-C). The first frame component 110 may include one or more wires. The one or more wires 350 may form the first frame component 110, the second frame component 120, and an intermediate portion (e.g., engagement section shown in FIG. 1). In other instances, the intermediate portion may include a graft material arranged between and connecting the first frame component 110 and the second frame component 120. The first frame component 110 may be arranged on a first side of the intermediate portion and the second frame component 120 may be arranged on a second side of the intermediate portion.

The first frame component 110 may include a plurality of contact features 240, one of which is highlighted in FIG. 8 for ease of illustration. In instances where an implantable device includes the first frame component 110 and the second frame component 120, the contact features 240 of the first frame component 110 may be a first plurality of contact features 240 and the contact features 240 of the second frame component 120 may be a second plurality of contact features 240. The contact features 240, in certain instances, may include a plurality of outer apices 242 and a plurality of inner apices 244 arranged between each of the plurality of contact features 240. In instances where an implantable device includes the first frame component 110 and the second frame component 120, the plurality of outer apices 242 and the plurality of inner apices 244 of the first frame component 110 may be a first plurality of outer apices 242 and the plurality of inner apices 244 and the plurality of outer apices 242 and the plurality of inner apices 244 of the second frame component 120 may be a plurality of second outer apices 242 and a plurality of second inner apices 244. In certain instances, a single frame component 110 may be used to anchor a sensor 500 with the adjacent ones of the contract features 240 being arranged on opposite sides of tissue.

In certain instances, the portions of the one or more wires 350 that form the first frame component 110 are uncoupled from portions of the one or more wires 350 that form the second frame component 120. In addition, the one or more wires 350 may define a plurality of engagement features for mating engagement with a sensor, such as with one or more recessed portions of the sensor. The engagement features may be arranged in a complementary pattern to the plurality of recessed portions. As shown, the one or more wires 350 may form a first engagement section 854 forming a boundary extending between the plurality of first inner apices 244 configured to capture at least a portion of the sensor within the first frame component 110 (e.g., by engaging recessed portions in the sensor 500 as described in detail below with reference to FIGS. 11-12). The apices 242, 244 may be engagement features. The second frame component 120 may also include a second engagement section 854 formed by portions of the one or more wires that may also be configured to capture at least a portion of the sensor within the second frame component 120 (e.g., by engaging recessed portions in the sensor 500 as described in detail below with reference to FIGS. 11-12). In certain instances, the first engagement section 854 and/or the second engagement section 854 may have approximately a circular shape. In addition. the ends of the wires 350 or wire 350 not touching may lessen or eliminate signal interference for the sensor 500. The first engagement section 854 and/or the second engagement section 854 may be configured to limit rotational freedom of the sensor 500 when anchored within the anchor 400. In certain instances and as is shown, the first engagement section 854 and/or the second engagement section 854 may include additional winding (e.g., a stored length) of the one or more wires that facilitates capture of the sensor 500 and translation of the sensor 500 if desired (e.g., by engaging recessed portions in the sensor 500 as described in detail below with reference to FIGS. 11-12).

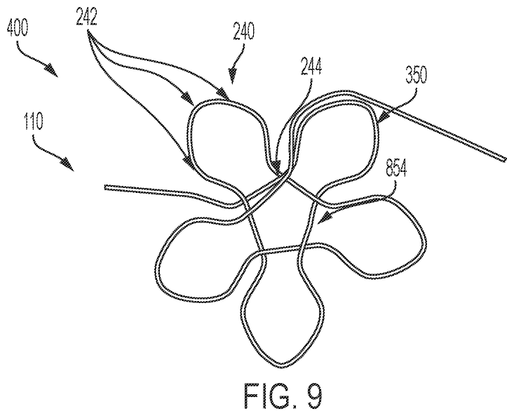

FIG. 9 is another example anchor for coupling to a sensor in accordance with an embodiment. The anchor 400 may anchor a sensor within a patient. The first frame component 110 shown in FIG. 9 may be the first frame component 110 and/or the second frame component 120 (e.g., as shown in FIGS. 1, 10A-C). The first frame component 110 may include one or more wires. The one or more wires 350 may form the first frame component 110, the second frame component 120, and an intermediate portion (e.g., engagement section shown in FIG. 1). In other instances, the intermediate portion may include a graft material arranged between and connecting the first frame component 110 and the second frame component 120. The first frame component 110 may be arranged on a first side of the intermediate portion and the second frame component 120 may be arranged on a second side of the intermediate portion.

The first frame component 110 may include a plurality of contact features 240, one of which is highlighted in FIG. 9 for ease of illustration. In instances where an implantable device includes the first frame component 110 and the second frame component 120, the contact features 240 of the first frame component 110 may be a first plurality of contact features 240 and the contact features 240 of the second frame component 120 may be a second plurality of contact features 240. The contact features 240, in certain instances, may include a plurality of outer apices 242 and a plurality of inner apices 244 arranged between each of the plurality of contact features 240. In instances where an implantable device includes the first frame component 110 and the second frame component 120, the plurality of outer apices 242 and the plurality of inner apices 244 of the first frame component 110 may be a first plurality of outer apices 242 and the plurality of inner apices 244 and the plurality of outer apices 242 and the plurality of inner apices 244 of the second frame component 120 may be a plurality of second outer apices 242 and a plurality of second inner apices 244. In certain instances, a single frame component 110 may be used to anchor a sensor 500 with the adjacent ones of the contract features 240 being arranged on opposite sides of tissue.

In certain instances, the portions of the one or more wires 350 that form the first frame component 110 are uncoupled from portions of the one or more wires 350 that form the second frame component 120. In addition, the one or more wires 350 may also form a first engagement section 854 forming a boundary extending between the plurality of first inner apices 244 (or contact features 244) configured to capture at least a portion of the sensor within the first frame component 110. The second frame component 120 may also include a second engagement section 854 formed by portions of the one or more wires that may also be configured to capture at least a portion of the sensor within the second frame component 120 (e.g., by engaging recessed portions in the sensor 500 as described in detail below with reference to FIGS. 11-12). In certain instances, the first engagement section 854 and/or the second engagement section 854 may have approximately a polygonal shape. The sides of the polygonal shape may define a plurality of engagement features for mating engagement with a sensor, such as with a plurality of recessed portions of the sensor. In various examples, the plurality of engagement features may be arranged in a complementary pattern to the plurality of recessed portions of the sensor (e.g., each having 2, 3, 5, etc. sides or portions for mating engagement with one another). The first engagement section 854 and/or the second engagement section 854 may be configured to limit rotational freedom of the sensor 500 when anchored within the anchor 400 (e.g., as shown in FIG. 11). In addition. the ends of the wires 350 or wire 350 not touching may lessen or eliminate signal interference for the sensor 500. The apices 242, 244 may be engagement features.

FIG. 10A is an example anchor 400 and a sensor 500 coupled to the anchor in accordance with an embodiment. As discussed in detail above, the anchor 400 may include a first frame component 110, a second frame component 120, and an engagement section 130 (or intermediate portion) configured to engage and anchor the sensor 500 within the anchor 400. The engagement section 130 may frictionally engage the sensor 500 to maintain the sensor 500 within the anchor 400. The engagement section 130 may include a structure or features that are configured to engage the sensor 500 as is shown. The engagement section 130 may be formed by portions of wires 350 (or struts), a graft material, or a combination of wires 350 and graft. The engagement section 130 may include an undulating wire that is configured to engage the sensor 500.

In certain instances, the first frame component 110 and the second frame component 120 are convexly curved inwardly relative to the engagement section 130. The curved frame components 110, 120 may facilitate contact with a target tissue location and also lessen or eliminate the opportunity for obstructive tissue growth along the curved frame components 110, 120 that could interfere with sensor 500 performance. The first frame component 110 and the second frame component 120 having a concave curvature may also allow for the anchor 400 to implant with a varying range of tissue thicknesses. The first frame component 110 and the second frame component 120 may be configured to maintain contact with tissue and may transition from a curved configuration to a flat configuration to better contact a tissue surface. In addition, the curved configuration of the frame component 110 and the second frame component 120 may prevent prolapse and/or thrombus formation by maintaining contact with a tissue surface. The curvature lessens the opportunity of a stagnant space to form that could fill with blood and cause thrombus. Further, the curved first frame component 110 and the second frame component 120 are configured to maintain a separation between one another as shown. In addition, the sensor 500 may include one or more recessed portions, as is shown, and the engagement section 130 may be configured to engage the one or more recessed portions in the sensor 500 with an interference fit to hold the sensor 500 within the anchor 400.

FIG. 10B is an example anchor 400 and a sensor 500 coupled to the anchor in accordance with an embodiment. As discussed in detail above, the anchor 400 may include a first frame component 110, a second frame component 120, and an engagement section 130 (or intermediate portion) configured to engage and anchor the sensor 500 within the anchor 400. The engagement section 130 may include a structure or features that are configured to engage the sensor 500 as is shown. The engagement section 130 may be formed by portions of wires 350 (or struts), a graft material, or a combination of wires 350 and graft. The engagement section 130 may be separate (or without wires 350 connecting) from the frame components 110, 120 and also be configured to engage the sensor 500. The engagement section 130 may be formed of a graft component to separate wire portions that form the frame components 110, 120 with a wire or strut element (shown) coupled or attached to the membrane of the engagement section 130 to engage and anchor the sensor 500.

In certain instances, the first frame component 110 and the second frame component 120 are convexly curved inwardly relative to the engagement section 130. The curved frame components 110, 120 may facilitate contact with a target tissue location and also lessen or eliminate the opportunity for obstructive tissue growth along the curved frame components 110, 120 that could interfere with sensor 500 performance. The first frame component 110 and the second frame component 120 having a concave curvature may also allow for the anchor 400 to implant with a varying range of tissue thicknesses. The first frame component 110 and the second frame component 120 may be configured to maintain contact with tissue and may transition from a curved configuration to a flat configuration to better contact a tissue surface. In addition, the curved configuration of the frame component 110 and the second frame component 120 may prevent prolapse and/or thrombus formation by maintaining contact with a tissue surface. The curvature lessens the opportunity of a stagnant space to form that could fill with blood and cause thrombus.

In certain instances, the anchor 400, as discussed herein, may include biocompatible low conductivity materials (e.g., biocompatible plastics, bioabsorbable materials) that avoid signal transmission interference design challenges associated with metals. Further, in addition to nitinol, non-shape memory metals and alloys such as stainless steel or cobalt chromium, can be used. Metal composites such as nitinol with a platinum core may be used to increase radiopacity. The materials of the anchor 400 may be insulated by coating, wrapping, jacketed, or infusing the metals with thick oxide layers to eliminate signal interference. In addition, the sensor 500 may include one or more recessed portions, as is shown, and the engagement section 130 may be configured to engage the one or more recessed portions in the sensor 500 with an interference fit to hold the sensor 500 within the anchor 400.

FIG. 10C is an example anchor 400 and a sensor 500 coupled to the implantable medical device in accordance with an embodiment. As discussed in detail above, the anchor 400 may include a first frame component 110 and a second frame component 120.

In certain instances, the first frame component 110 and the second frame component 120 are convexly curved inwardly relative to one another, The curved frame components 110, 120 may facilitate contact with a target tissue location and also lessen the opportunity for obstructive tissue growth along the curved frame components 110, 120 that could interfere with sensor 500 performance. The first frame component 110 and the second frame component 120 having a concave curvature may also allow for the anchor 400 to implant with a varying range of tissue thicknesses. The first frame component 110 and the second frame component 120 may be configured to maintain contact with tissue and may transition from a curved configuration to a flat configuration to better contact a tissue surface. In addition, the curved configuration of the frame component 110 and the second frame component 120 may prevent prolapse and/or thrombus formation by maintaining contact with a tissue surface. The curvature lessens the opportunity of a stagnant space to form that could fill with blood and cause thrombus. Further, the curved first frame component 110 and the second frame component 120 are configured to maintain a separation between one another as shown.

Although the first frame component 110 and/or the second frame component 120 may incorporate a curved, or “umbrella” like configuration as shown in FIGS. 10A to 10C, it should be appreciated that such a feature is not necessary to benefit from the ability of the various embodiments described herein to maintain spacing between the first and second frame components 120. In particular, by being maintained at desired spacing from one another, and relatively to a longitudinal position on the sensor, the first and second frame components 110, 120 are better able ensure desirable apposition with tissue received between the first and second frame components. If the first and second frame components 110, 120 are insufficiently spaced from one another, or if such spacing is not sufficiently maintained, the first and/or second frame components 110, 120 may take on a cupped shape, with the edges of the first and second frame components flaring or curling away from the tissue to which it would otherwise be engaged. This, or more generally insufficient engagement between the first and second frame components 110, 120 and the tissue therebetween, can result in the opportunity of a stagnant space to form that could fill with blood and cause thrombus. In various examples, the first and second frame components are maintained at a spacing of about 6 mm, between 3 mm and 9 mm, or at another desired spacing. In the context of septal positioning (e.g., atrial septum) of the device, it has been postulated that by ensuring a spacing of about 6 mm, sufficient apposition will be achieved with the septal wall for a variety of typical septum thicknesses.

FIG. 11 is an example sensor 500 having recessed portions 1200 spaced about a circumference of the sensor 500. There may be a plurality of recessed portions 1200 spaced about a circumference of the sensor 500 in certain instances. The recessed portions 1200 may be rectangular in shape and etched or scored into the surface of the sensor 500, for example. The recessed portions 1200 may be in one or more locations relative to a length of the sensor 500 as is shown.

In certain instances, plurality of recessed portions 1200 (or engagement sections of the sensor 500) may be a first set of recessed portions 1200a and a second set of recessed portions 1200b. The first set of recessed portions 1200a may be configured to engage a portion of a first frame component of an anchor 400 and the second set of recessed portions 1200b may be configured to engage a portion of a second frame component of an anchor 400 (e.g., anchors 400 shown in FIGS. 2-4 and 6-9. In certain instances, the plurality of inner apices of the first component are configured to engage the first set of recessed portions 1200a and the plurality of inner apices (or contact features) of the second frame component are configured to engage the second set of recessed portions 1200b in a mating relationship.

As shown, the first second set of recessed portions 1200a and the second set of recessed portions 1200b are spaced apart from one another relative to a length of the sensor 500. Thus, and in certain instances, the second set of recessed portions 1200a and the second set of recessed portions 1200b may be configured to maintain a spaced relationship between the first frame component and the second frame component. In addition, the second set of recessed portions 1200a and the second set of recessed portions 1200b may be configured to maintain a rotational orientation of the first and second frame components relative to the sensor 500. The

FIG. 12 is another sensor 500 having recessed portions 1200 spaced about a circumference of the sensor 500. There may be a plurality of recessed portions 1200 spaced about a circumference of the sensor 500 in certain instances. The recessed portions 1200 may be rectangular in shape and etched or scored into the surface of the sensor 500, for example. The recessed portions 1200 may be in one or more locations relative to a length of the sensor 500 as is shown. In certain instances, the sensor 500 may also include one or more slots 1302 that may be a channel for end portions of wires that form the anchors 400 as discussed in detail above. The one or more slots 500 may facilitate maintaining separate of the wires to lessen or eliminate signal interference and lessen or eliminate potential for rotation of the sensor 500 relative to the anchor 400.

In certain instances, plurality of recessed portions 1200 (or engagement sections of the sensor 500) may be a first set of recessed portions 1200a and a second set of recessed portions 1200b. The first set of recessed portions 1200a may be configured to engage a portion of a first frame component of an anchor 400 and the second set of recessed portions 1200b may be configured to engage a portion of a second frame component of an anchor 400 (e.g., anchors 400 shown in FIGS. 2-4 and 6-9. In certain instances, the plurality of inner apices of the first component are configured to engage the first set of recessed portions 1200a and the plurality of inner apices (or contact features) of the second frame component are configured to engage the second set of recessed portions 1200b in a mating relationship.

As shown, the first second set of recessed portions 1200a and the second set of recessed portions 1200b are spaced apart from one another relative to a length of the sensor 500. Thus, and in certain instances, the second set of recessed portions 1200a and the second set of recessed portions 1200b may be configured to maintain a spaced relationship between the first frame component and the second frame component. In addition, the second set of recessed portions 1200a and the second set of recessed portions 1200b may be configured to maintain a rotational orientation of the first and second frame components relative to the sensor 500.

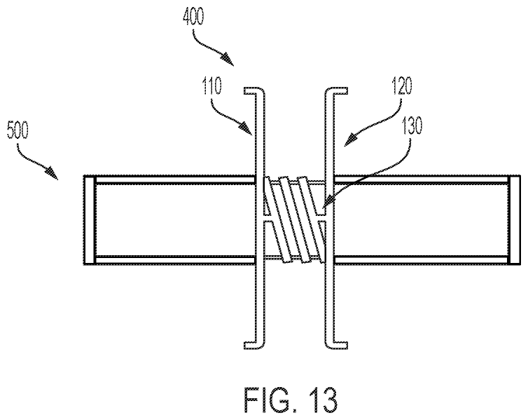

FIG. 13 is an example anchor 400 and a sensor 500 coupled to the anchor in accordance with an embodiment. As discussed in detail above, the anchor 400 may include a first frame component 110, a second frame component 120, and an engagement section 130 (or intermediate portion) configured to engage and anchor the sensor 500 within the anchor 400. The engagement section 130 may include a structure or features that are configured to engage the sensor 500 as is shown. The engagement section 130 may be formed by portions of wires 350 (or struts), a graft material, or a combination of wires 350 and graft. The engagement section 130 may be separate (or without wires 350 connecting) from the frame components 110, 120 and also be configured to engage the sensor 500. The engagement section 130 may be formed of a graft component to separate wire portions that form the frame components 110, 120 with a wire or strut element (shown) coupled or attached to the membrane of the engagement section 130 to engage and anchor the sensor 500.

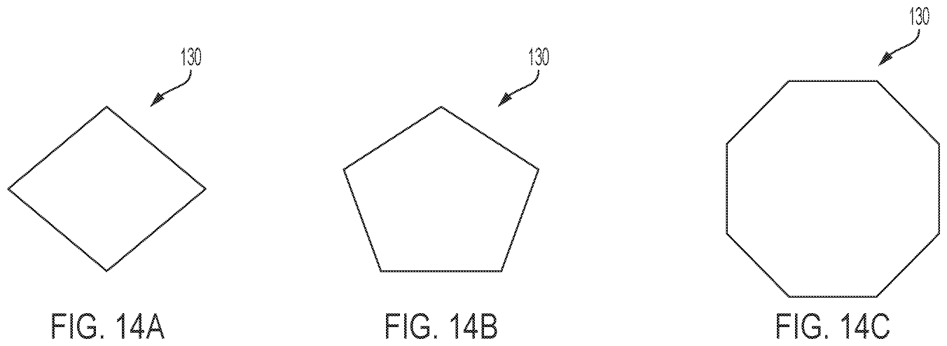

In certain instances, the engagement section 130 may include a perimeter that is polygonal as shown in FIGS. 14A-C, which show example perimeters for an engagement section 130. The polygonal shapes, having even or odd numbers of sides, may be configured to engage recessed portions in the sensor 500 (e.g., as shown in FIGS. 11-12). In certain instances, the number of recessed portions may be equal to the number of sides of the polygonal shape of the engagement section 130. Apices of wires or cut tubes may form the perimeter shape of the engagement section 130 (e.g., as described above with reference to FIGS. 6-9). In addition, the sensor 500 may include one or more recessed portions, as is shown, and the engagement section 130 may be configured to engage the one or more recessed portions in the sensor 500 with an interference fit to hold the sensor 500 within the anchor 400. As discussed above, the one or more recessed portions in the sensor 500 may be etched in the material of the sensor 500, removed from the material of the sensor 500, molded into the material of the sensor 500, or deposited on the material of the sensor 500, for example.

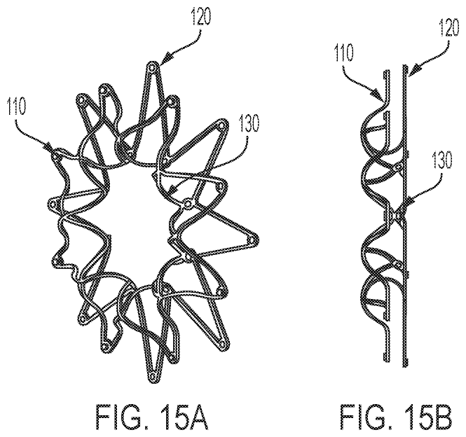

FIGS. 15A-B are perspective and side views of an example anchor configured to engage a sensor in accordance with an embodiment. As discussed in detail above, the anchor 400 may include a first frame component 110, a second frame component 120, and an engagement section 130 (or intermediate portion) configured to engage and anchor the sensor 500 within the anchor 400. The engagement section 130 may include a structure or features that are configured to engage the sensor 500 as is shown. The engagement section 130 may be formed by portions of wires 350 (or struts), a graft material, or a combination of wires 350 and graft. The engagement section 130 may be separate (or without wires 350 connecting) from the frame components 110, 120 and also be configured to engage the sensor 500. The engagement section 130 may be formed of a graft component to separate wire portions that form the frame components 110, 120 with a wire or strut element (shown) coupled or attached to the membrane of the engagement section 130 to engage and anchor the sensor 500.

In certain instances, the engagement section 130 may include a perimeter that is polygonal as shown in FIGS. 14A-C, which show example perimeters for an engagement section 130. The polygonal shapes, having even or odd numbers of sides, may be configured to engage recessed portions in the sensor 500 (e.g., as shown in FIGS. 11-12). In certain instances, the number of recessed portions may be equal to the number of sides of the polygonal shape of the engagement section 130. Apices of wires or cut tubes may form the perimeter shape of the engagement section 130 (e.g., as described above with reference to FIGS. 6-9).

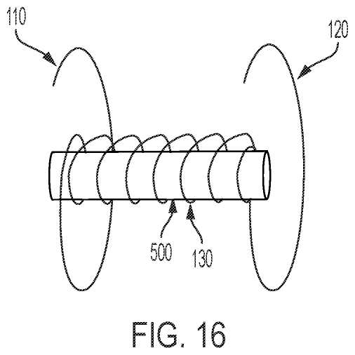

FIG. 16 is an example anchor 400 and a sensor 500 coupled to the anchor in accordance with an embodiment. As discussed in detail above, the anchor 400 may include a first frame component 110, a second frame component 120, and an engagement section 130 (or intermediate portion) configured to engage and anchor the sensor 500 within the anchor 400. The engagement section 130 may include a structure or features that are configured to engage the sensor 500 as is shown. The engagement section 130 may be formed by portions of wires 350 (or struts), a graft material, or a combination of wires 350 and graft. The engagement section 130 may be separate (or without wires 350 connecting) from the frame components 110, 120 and also be configured to engage the sensor 500. The engagement section 130 may be formed of a graft component to separate wire portions that form the frame components 110, 120 with a wire or strut element (shown) coupled or attached to the membrane of the engagement section 130 to engage and anchor the sensor 500.

In certain instances, the engagement section 130 may include a perimeter that is polygonal as shown in FIGS. 14A-C, which show example perimeters for an engagement section 130. The polygonal shapes, having even or odd numbers of sides, may be configured to engage recessed portions in the sensor 500 (e.g., as shown in FIGS. 11-12). In certain instances, the number of recessed portions may be equal to the number of sides of the polygonal shape of the engagement section 130. Apices of wires or cut tubes may form the perimeter shape of the engagement section 130 (e.g., as described above with reference to FIGS. 6-9).

A membrane material, discussed herein, may include a fluoropolymer, such as a polytetrafluoroethylene (PTFE) polymer or an expanded polytetrafluoroethylene (ePTFE) polymer. In some instances, the membrane material may be formed of, such as, but not limited to, a polyester, a silicone, a urethane, a polyethylene terephthalate, or another biocompatible polymer, or combinations thereof. In some instances, bioresorbable or bioabsorbable materials may be used, for example a bioresorbable or bioabsorbable polymer. In some instances, the membrane material can include Dacron, polyolefins, carboxy methylcellulose fabrics, polyurethanes, or other woven, non-woven, or film elastomers. The membrane material (e.g., when a fluoropolymer such as PTFE or ePTFE) may be configured to wet out (e.g., absorb blood components and/or fluid) after contact with blood while lessening the opportunity for signal interference. The fluoropolymer membrane may lessen or eliminate conduction that may affect signal performance even after wetting out.

In addition, nitinol (NiTi) may be used as the material of the frame (and any of the frames discussed herein), but other materials such as, but not limited to, stainless steel, L605 steel, polymers, MP35N steel, polymeric materials, Pyhnox, Elgiloy, or any other appropriate biocompatible material, and combinations thereof, can be used as the material of the frame. The super-elastic properties and softness of NiTi may enhance the conformability of the frame elements. In addition, NiTi can be shape-set into a desired shape. That is, NiTi can be shape-set so that the frame tends to self-expand into a desired shape when the frame is unconstrained, such as when the frame is deployed out from a delivery system.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. A sensor assembly for placement across the atrial septum of a patient, the sensor assembly comprising:
a sensor having one or more recessed portions in an outer surface of the sensor;
an anchor comprising:
a first frame component; and
a second frame component spaced from the first frame component,
wherein the anchor is engaged with the one or more recessed portions in the outer surface of the sensor in a mating relationship such that the first frame component and the second frame component are rotationally fixed relative to the sensor.

2. The sensor assembly of claim 1, wherein the anchor is engaged with the one or more recessed portions such that the first and second frame components are maintained at a selected spacing relative to one another.

3. The sensor assembly of claim 1, wherein the anchor further comprises an engagement section coupled to at least one of the first frame component and the second frame component, the engagement section having an inner profile that is engaged with the one or more recessed portions in the outer surface of the sensor.

4. The sensor assembly of claim 3, wherein the inner profile of the engagement section has a polygonal shape defining a plurality of engagement features for mating engagement with the one or more recessed portions in the outer surface of the sensor.

5. The sensor assembly of claim 3, wherein the inner profile of the engagement section has a circular shape.

6. The sensor assembly of claim 3, wherein the inner profile of the engagement section is defined by a membrane material.

7. The sensor assembly of claim 3, wherein the engagement section is a first engagement section associated with the first frame component, and further wherein the anchor further comprises a second engagement section associated with the second frame component.

8. The sensor assembly of claim 3, wherein the anchor further comprises one or more wires forming the engagement section, the inner circumference of the engagement section being defined by at least one turn of the one or more wires.

9. The sensor assembly of claim 3, wherein the one or more recessed portions of the sensor include a first set of recessed portions and a second set of recessed portions, and the first frame component is coupled to the first set of recessed portions and the second frame component is coupled to the second set of recessed portions.

10. The sensor assembly of claim 9, wherein the first frame component comprises a first plurality of engagement features defining a first engagement section of the anchor, and the second frame component comprises a second plurality of engagement features defining a second engagement section of the anchor, and further wherein the first plurality of engagement features engages the first set of recessed portions in a mating relationship and the second plurality of engagement features engages the second set of recessed portions in a mating relationship.

11. The sensor assembly of claim 10, wherein the first plurality of engagement features are arranged in a complementary pattern to the first set of recessed portions and the second plurality of engagement features are arranged in a complementary pattern to the second set of recessed portions.

12. A sensor assembly for placement across the atrial septum of a patient, the sensor assembly comprising:
a sensor having one or more recessed portions in an outer surface of the sensor;
an anchor comprising:
a first frame component; and
a second frame component spaced from the first frame component,
wherein the anchor is engaged with the one or more recessed portions in the outer surface of the sensor in a mating relationship such that the first frame component and the second frame component are maintained at a selected spacing relative to one another.

13. The sensor assembly of claim 12, wherein the anchor further comprises a first engagement section associated with the first frame component that is configured to capture at least a portion of the sensor and engage the one or more recessed portions and a second engagement section associated with the second frame component that is configured to capture at least another portion of the sensor and engage the one or more recessed portions.

14. The sensor assembly of claim 13, wherein the one or more recessed portions in the outer surface of the sensor includes a first set of recessed portions to which the first frame component is coupled and a second set of recessed portions to which the second frame component is coupled such that the first frame component and the second frame component are maintained at the selected spacing relative to one another.

15. The sensor assembly of claim 12, wherein the first and second engagement sections each comprise at least one wire.

16. The sensor assembly of claim 15, wherein the at least one wire includes a continuous wind pattern through the first frame component and the second frame component.

17. The sensor assembly of claim 13, wherein the first and second engagement sections are engaged with the one or more recessed portions in the outer surface of the sensor in a mating relationship such that a rotational orientation of the first and second frame components is maintained.

18. A method of managing treatment of a patient with congestive heart failure, the method comprising:
arranging a first frame component of an anchor on a first side of the atrial septum, the anchor being in mating engagement with a plurality of recessed portions in an outer surface of a sensor; and
arranging a second frame component of the anchor on a second side of the atrial septum such that the sensor is arranged to monitor pressure in the left atrium, the right atrium, or both the left atrium and the right atrium.

19. The method of claim 18, wherein the anchor comprises an engagement section coupled to at least one of the first frame component and the second frame component, and further wherein the engagement section is in mating engagement with the plurality of recessed portions in the outer surface of the sensor such that a rotational orientation of the first and second frame components relative to the sensor is maintained.

20. The method of claim 18, wherein the engagement section is in mating engagement with the plurality of recessed portions in the outer surface of the sensor such that a spacing between the first and second frame components is maintained.

21. The method of claim 18, wherein the plurality of recessed portions of the sensor include a first set of recessed portions and a second set of recessed portions, and the first frame component is coupled to the first set of recessed portions and the second frame component is coupled to the second set of recessed portions.

22. A sensor assembly for placement across the atrial septum of a patient, the sensor assembly comprising:
a sensor having one or more recessed portions in an outer surface of the sensor;
a first frame component including a plurality of first contact features and a first engagement section configured to capture at least a portion of the sensor and engage the one or more recessed portions to limit rotational freedom of the sensor relative to the first frame component; and
a second frame component including a plurality of second contact features and a second engagement section configured to capture at least a portion of the sensor and engage the one or more recessed portions to limit rotational freedom of the sensor relative to the second frame component.

23. The sensor assembly of claim 22, wherein the first engagement section is defined by a plurality of first inner apices that approximate a shape that includes a first circumference that is about equal to a circumference of the sensor and the second engagement section is defined by a plurality of second inner apices that approximate a shape that includes a second circumference that is about equal to a circumference of the sensor.

24. The sensor assembly of claim 22, wherein the first and second engagement sections are configured to maintain a spacing between the first and second frame components.

25. The sensor assembly of claim 22, wherein the engagement section includes a plurality of engagement features arranged in a complementary pattern to the plurality of recessed portions.

26. The sensor of claim 23, wherein the one or more recessed portions in the exterior surface of the sensor are configured to engage with the plurality of first inner apices and the plurality of second inner apices.

27. A sensor assembly for placement across the atrial septum of a patient, the sensor assembly comprising:
a sensor having one or more recessed portions in an outer surface;
a first frame component including a plurality of first contact features;
a second frame component including a plurality of second contact features; and
an intermediate portion including a graft material arranged between and connecting the first frame component and the second frame component, the intermediate portion configured to engage the one or more recessed portions in a mating relationship to couple the first and second frame components to the sensor.

28. The sensor assembly of claim 27, wherein the first frame component includes a plurality of first inner apices that approximate a shape that includes a first circumference that is greater than a circumference of the sensor and the second frame component includes a plurality of second inner apices that approximate a shape that includes a second circumference that is greater than a circumference of the sensor.

29. A sensor assembly for placement across the atrial septum of a patient, the sensor assembly comprising:
a sensor having one or more recessed portions in an outer surface;
a first frame component including a plurality of first contact features and a first engagement section forming a first inner boundary configured to engage the recessed portions in mating engagement to limit rotational freedom of the sensor relative to the first frame component; and
a second frame component arranged opposite the first frame component on the sensor, the second frame component including a plurality of second contact features and a second engagement section forming a second inner boundary configured to engage the recessed portions in mating engagement to limit rotational freedom of the sensor relative to the second frame component.

30. The sensor assembly of claim 29, wherein the one or more recessed portions includes a first set of recessed portions and a second set of recessed portions, and further wherein the first engagement section includes a plurality of engagement features arranged in a complementary pattern to the first set of recessed portions and the second engagement section includes a plurality of engagement features arranged in a complementary pattern to the second set of recessed portions.

31. The sensor assembly of claim 29, further including an intermediate portion including a graft material arranged between and connecting the first frame component and the second frame component.

32. The sensor assembly of claim 29, wherein the first engagement section has an approximately circular shape and the second engagement section has an approximately circular shape.

33. A sensor assembly for placement across the atrial septum of a patient, the sensor assembly comprising:
a sensor having one or more recessed portions in an outer surface of the sensor;
a first frame component including a first engagement section forming a boundary configured to capture at least a portion of the sensor and engage the one or more recessed portions; and
a second frame component including a second engagement section forming a boundary configured to capture at least a portion of the sensor and engage the one or more recessed portions, the first and second engagement sections being configured to maintain a space between the first and second frame components.

34. The sensor assembly of claim 33, wherein the first engagement section has an approximately polygonal shape and the second engagement section has an approximately polygonal shape.

36. A sensor assembly for placement across the atrial septum of a patient, the sensor assembly comprising:
a sensor having one or more recessed portions in an outer surface;
an anchor comprising:
an engagement section configured to engage the one or more recessed portions in the sensor with an interference fit to hold the sensor within the anchor; and
a first frame component and a second frame component, the engagement section configured to maintain a space between the first and second frame components.
